(19) United States
(12) Patent Application Publication
(10) Pub. No.: US 2003/0220414 A1
(43) Pub. Date: Nov. 27, 2003

(54) Biocompatible cement compositions and method for filling a skeletal cavity using said cement compositions

(76) Inventors: Niklas Axen, Jarlasa (SE); Leif Hermansson, Uppsala (SE); Dan Markusson, Vaxjo (SE); Lennart Pedersen, Loftahammar (SE)

Correspondence Address: YOUNG & THOMPSON, 745 SOUTH 23RD STREET 2ND FLOOR, ARLINGTON, VA, 22202

(21) Appl. No.: 10/405,359

(22) Filed: Apr. 3, 2003

(30) Foreign Application Priority Data
Apr. 4, 2002 (SE) 0201052-8

(51) Int. Cl. A61F 2/28

(52) U.S. Cl. 523/116; 623/23.62

(57) ABSTRACT

Biocompatible cement compositions in a hardened state for filling an orthopaedic cavity and fixating a medical implant in the skeletal bone, by mixing fixation grains (granules) with a biocement slurry or paste, either inside or outside an orthopaedic cavity. A medical implant can be inserted into the grains either before or after the addition of the biocement slurry or paste. The biocompatible cement compositions achieve both high initial fixation strength, as well as a fixation providing long-term stability and biocompatibility, without any negative health effects. The biocompatible cement compositions can suitably be used for filling orthopaedic cavities due to for example osteoporosis, cancer, fractures or other types of bone defects, and can also be used for fixating general orthopaedic and dental implants.

### THE FIELD OF THE INVENTION

The present invention relates to biocompatible cement compositions applicable in the orthopaedic and dental fields. More precisely the invention relates to biocompatible cement compositions for treating cavities in the skeletal bone to achieve a biocompatible and mechanically strong result. Alternatively, the biocompatible cement compositions may be used for fixation of orthopaedic implants such as hip and knee joints, or dental implants, in cavities created in the skeletal bone. The present invention also relates to a method for filling such a cavity with said biocompatible cement compositions.

### BACKGROUND OF THE INVENTION

Orthopaedic and dental biocements

In some fields of surgery, particularly orthopaedics and odontology, in-situ hardening biomaterials, here referred to as biocements, are used in several contexts. The materials are used for fixation of joint implants, e.g. hips-joints, to strengthen osteoporotic bone, to replace cancerous bone, for fracture treatment as well as for dental applications such as tooth and root fillings. These cements may be prepared in a clinical environment, moulded by the surgeon to desired shape and even injected to a selected position in the body, where they cure to a solid body.

The most established orthopaedic cements are based on the polymer polymethylmethacrylate (PMMA), with the addition of various fillers to optimise mechanical or other properties. This group of cements is mainly used for anchoring hip-joint prostheses in the femoral and pelvic bones, or for the corresponding anchoring of knee joints.

PMMA-cements have favourable mechanical properties, but poor biocompatibility. They also suffer from disadvantages such as excessive heat generation during hardening (exceeding 50° C., thus risking to cause tissue necrosis) and shrinkage during polymerisation (approximately 2-5%), which impairs the mechanical anchoring in the adjacent bone and the possibility of early loading of the prosthesis. There is also a risk of deformation of the cement over time due to creep. Still PMMA-based materials are well established since decades, both for orthopaedic and dental applications.

In addition to the polymer-based cements, there are in-situ hardening cements based on ceramic components. Examples of ceramic biocement products are: Norian SRS® and Biobon®. In general, ceramic cements are more biocompatible than those of PMMA. However, they often suffer from inferior mechanical strength. The manufacturers of Norian® and Biobon® provide compressive strength values around 40 and 50 MPa, respectively, much lower values that for natural bone.

A novel biocement based on the substance calcium aluminate is described in the pending patent application SE-0 104 441-1 with the title “Ceramic material and process for manufacturing”. Compared to other ceramic cements, the novel material has superior mechanical properties, and a high degree of chemical and mechanical stability in the body environment. Compared to PMMA cements this novel cement hardens at lower temperature and possesses higher biocompatibility.

For the fixation of joint implants, the polymer-based cements are dominating. Such implants may, however, also be used without cement, so called cement-less implants. This requires a direct bond between the implant and the bone tissue.

Filling of orthopaedic cavities and attachment of implants using packed grains An alternative technique for the attachment of implants in the skeletal bone, e.g. hip-implants, is disclosed in Swedish patent SE-462 638.

This method may essentially also be used for filling general cavities in the skeletal bone (e.g. created when cancerous bone is removed), or for strengthening of osteoporotic bone.

According to SE-462 638, the spacing or cavity between the prosthesis and the bone wall is filled with grains (here called fixation grains), which are described as essentially non-elastic and preferably irregular in shape and preferably porous. Several materials are suggested for the grains, both metals and ceramics. Grains of titanium are however preferred. Grain sizes in the range of 0.1-2 mm are suggested.

In the method according to SE-462 638, the cavity is first filled with grains. Thereafter an implant is inserted into the grain volume, followed by application of a vibrating tool (vibrator) on the implant. This makes the implant vibrate and the vibrations are transferred from the implant to the grains, creating a “floating” bed as the grains oscillate against each other. With the active vibrator pressed against the implant, the implant can be inserted into the grain volume. As the vibrator is turned off or removed from the implant, the grains interlock and the implant is anchored.

The applied vibrations thus both contribute to increase the number of grains per volume unit, and also to make the insertion of an implant into the grains possible.

A major advantage with the described technique is the immediate fixation of the implant. Another advantage is that the spacing between implant and bone is filled with a biocompatible implant material (the titanium grains instead of PMMA bone cement) . It is also claimed that the porous structure created between implant and the bone wall triggers bone regeneration, i.e. new bone tissue grows in-between the grains.

A disadvantage with the technique is the low early strength of the fixation, before new bone tissue has infiltrated the grains. Presumably, also the long-term strength is lower than for a conventionally cemented or cement-less implant.

SE-462 638 also mentions that the spacing between the grains may be filled with biological material, e.g. ground or crushed bone, to enhance the regeneration of tissue. The technique can also be used to attach dental implants.

It is also mentioned in the background to SE-462 638, that the grains may be locked to each other by using a binder. The binder may be added to the cavity, after or before the vibration, to lock (glue) the grains to each other. A suitable binder is however not suggested or described, and the use of a binder is not incorporated in the claims.

Hydraulic Biocements

Hydraulic cement is a type of ceramic material, for which the hardening process follows as a result of chemical reactions between ceramic powders and water, i.e. hydration. This group of so-called hydraulic cements include materials ranging from concrete based on Portland cement to special ceramics used in dentistry and orthopaedics.

Traditionally, cement processing involves preparation of the raw material by high temperature processing of selected minerals, grinding to fine powders, mixing of powder and water possibly together with additives controlling properties such as strength, rheology and hardening rate, followed by shaping/moulding of the powder-water fix, and finally hardening/solidification by hydration reactions. When water, or a water-based solution, is added to a powder of hydraulic cement, a hardening process starts due to hydration. As a result of the hydration, a new binding phase of hydrates is developed.

### SUMMARY OF THE INVENTION

In view of the drawbacks associated with the prior art biocompatible cement compositions used for filling orthopaedic cavities and for anchoring orthopaedic and dental implants in the skeletal bone, there is a need for biocompatible cement compositions with which both a mechanically strong initial fixation, making early loading of the implant possible, and long-term stability is obtained, and which only includes biocompatible materials.

The object of the present invention is to provide biocompatible cement compositions that can be used for filling cavities in the skeletal bone due to for example osteoporosis, cancer, fractures or other types of bone defects and which achieves both high initial fixation strength and long-term stability, and has no negative health effects. The present invention achieves this object with the features of the biocompatible cement composition defined in claim 1.

In another aspect of the invention, there is provided a method for filling such cavities using the biocompatible cement compositions as discussed below and securely fixating orthopaedic and dental implants in the skeletal bone.

The method and biocompatible cement compositions according to the present invention can suitably be used for filling orthopaedic cavities and fixating general orthopaedic and dental implants in the skeletal bone.

### DETAILED DESCRIPTION OF THE INVENTION

The present invention relates to biocompatible cement compositions applicable in the orthopaedic and dental fields. More precisely the invention relates to biocompatible cement compositions used for filling cavities in the skeletal bone with a biocompatible and mechanically strong substance and for fixating implants such as hip and knee joints or dental implants in the skeletal bone. Filling cavities includes completely and incompletely filling a cavity space.

The filling of orthopaedic cavities may for example be necessary for restorative purposes after damages to the bone caused by e.g. fractures, osteoporosis, or when cancerous bone needs to be removed and replaced. In the case of osteoporosis, cavities of particular interest are the interior of the vertebrae of the spine, and the cancellous bone of regions close to joints, e.g. knee and hip.

The inventive biocompatible cement compositions combine fixation grains of biocompatible materials with in-situ hardening biocements. With “biocompatible cement composition” we mean a cement composition having biocompatible properties and having been made by combining inert fixation grains and biocement. With “biocement” we mean the hardening phase of cement having biologically acceptable properties. The manufacturing of the biocompatible cement compositions according to the present invention comprises the following general steps:

First, a pre-created cavity is filled with comparatively large grains, which are packed by pressure or vibrations to completely fill the cavity and provide fixation to the implant. Secondly, the spacing between the grains is filled with a paste or slurry based on hydraulic biocement with considerably more fine-grained ingredients, which hardens in-situ and binds the fixation grains to each other. Alternatively, the orthopaedic cavity is filled with the fixation grains together with the biocement in one step.

The steps of the method of manufacturing the biocompatible cement compositions according to the present invention will now be described in more detail.

Before adding the present invention compositions, a suitable, clean and dry cavity is created. This is done using established surgical techniques. For the purpose of attaching a hip-joint, the cavity is the interior channel of the femoral bone. For the purpose of stabilizing a vertebra collapsed due to osteoporosis, the cavity is the spongy interior of the vertebra. The cavity may also be the result of removal of a cancerous segment of bone. The cavity is kept free from blood or other body fluids.

In a first step, the cavity is filled with grains. These grains should preferably be of a biocompatible material, e.g. titanium, as described e.g. in patent SE-462 638. Other metals like vitallium alloys of the Co—Cr—Mo—V system, stainless steels or Co—Cr alloys can also be used. Ceramic grains, e.g. alumina, zirconia, silicon nitride or materials from the group of ceramics referred to as SiAlONs, (ceramic compounds based on mixtures of silicon, aluminium, oxygen and nitrogen) may also be used. However, the embedding of the grains in biocompatible cement according to the present invention reduces the requirement on the grains in terms of biocompatibility, and opens up for a wider selection of grain materials. The grains may thus be selected from the group consisting of metals and alloys thereof, ceramics and polymers.

As is well known within the field, the hardening temperature of a biocement used in situ in the body must be controlled to prevent damage to the adjacent tissue. The use of fixation grains in the biocement slurry or paste also allows the use of cements, hydraulic or others, which develop heat during hardening. Compared to filling the entire cavity with cement alone, the heat generated by the cement during hardening is reduced in the method of the present invention, since a reduced amount of cement is used. The generated heat is reduced in proportion to the reduction of the amount of cement used.

Optionally, biological tissue, such as ground bone, can be added to the fixation grains, as described as an alternative procedure in the patent SE-462 638, to increase the rate of bone in-growth. However, the addition of ground bone or bone chips may affect the strength of the fixation.

The grains are compacted by pressure or vibrations as described in for example patent SE-462 638, in order to fill the entire cavity. As the grains are compacted, the volume that they occupy is reduced, wherefore additional grains may have to be added to compensate for the increased degree of compaction.

In a second step, the void volume between the grains is filled with a paste or slurry comprising hydraulic cement powder and water-based liquid. The grain bed may be completely filled with the slurry or paste using the vibrator in the manner described above.

Alternatively, the orthopaedic cavity may be filled in one step with a pre-made a biocompatible cement composition including both the fixation grains and the biocement. An implant may then be inserted into the cement slurry/paste either immediately after the filling is completed or after the slurry has been allowed to harden slightly.

According to another aspect of the present invention, there is also provided a method of fixating a medical implant in the skeletal bone, comprising the steps of filling a cavity with fixation grains, inserting a medical implant into the grains, and adding a biocement slurry or paste to the cavity filled with grains in order to lock them in position when allowing the biocement to harden.

In a preferred embodiment, the method also comprises applying vibrations to said implant in order to transfer vibrations to the grains and closely pack them. When said vibrations are interrupted, the grains interlock.

In a more preferred embodiment, the method comprises applying vibrations after the addition of the biocement, whereby the biocement is allowed to completely enter the void volume between the grains, thus reducing the degree of porosity in the hardened cement.

The medical implant used in these embodiments of the present invention can be made of a material selected from the group consisting of biocompatible materials, metals and alloys thereof, ceramics and polymers, but are preferably selected from the group consisting of biocompatible materials, such as titanium, vitallium alloys of the Co—Cr—Mo—V system, stainless steels, Co—Cr alloys.

The medial implants that can be used with the present invention can be selected from the group consisting of medical devices for implantation, artificial orthopedic devices, spinal implants, joint implants, attachment elements, bone nails, bone screws, or a bone reinforcement plates.

Biocompatible and mechanically strong cements suitable as binders for the purpose of locking the grains in position according to the present invention method are described below.

In one basic embodiment, the biocement according to the present invention only comprises calcium aluminate. This is hydraulic cement consisting essentially of phases from the CaO—Al2O3-system. A variety of phases belonging to this system are described in the literature, all of which are applicable on the present invention. Calcium aluminates are commercially available for example as the products Secar or Ternal White from LaFarge Aluminates. However, hydraulic cements of calcium silicates are also relevant to the invention, as well as cements of either or both of these substances with additions of property ameliorating additives. Cement based on calcium aluminate is preferred.

Phase systems based on hydrated calcium aluminate have unique properties. In comparison to other water binding ceramic systems, for example carbonates and sulphates of calcium, the aluminates are characterised by high chemical resistance, high strength and a relatively rapid hardening. The high strength of calcium aluminate cements is due to its high capacity of absorbing hydration water, which in turn results in low residual water content and low porosity. The low degree of porosity also increases the resistance to corrosion.

Among hydrating binding phase systems, calcium aluminate therefore has essential advantages as an implant material. The material hardens through reaction with water, which implies that the hardening process is not disturbed by the water-based body fluids. Before hardening, the material is well workable; it can be used both as slurry or paste. In the hardened condition the material possesses a unique combination of chemical inertness and mechanical strength, when compared to other hydrating compounds. For hardening above 30° C., stable hydrates form very quickly. This is of particular interest for implants, used at around 37° C. Also calcium silicates possess these properties to an acceptable degree.

Biocements based on calcium aluminates are e.g. described in the pending Swedish patent application SE-0 104 441-1 with the title “Ceramic material and process for manufacturing”. All substances covered by this pending patent application are suitable for use with the present invention.

The hydraulic cement powder grain size is preferably reduced in such a way that more than 50 vol.%, preferably more than 80 vol.%, and most preferably more than 90 vol.% of the powder comprises grains of a size within the range 0.5-20 microns. The preferred size is between 1 and 5 microns. This can be achieved by any conventional means and can be exemplified by ball milling.

Before preparing the biocement slurry or paste according to the invention, any residual water, organic material, or a combination thereof present in the powder (originating from processing, e.g. powder mixing, grain size reduction, or the like) should be removed. This can be achieved by any conventional means, such as heating of the powder at a sufficiently high temperature.

The properties of the biocement used in the present invention method may be improved with additives. These are described below.

A preferred composition of the cement is described in the pending Swedish pending patent application SE-0 104 441-1 with the title “Ceramic material and process for manufacturing”. In said patent application, in order to create a cement with lower content of aluminium, a filler material is added. As proposed in said application, calcium titanates, CaTiO3, or other variants where Ti may be substituted by Zr or Hf and Ca by Mg, Ca, Sr or Ba, in a perovskitic structure, are preferred for this purpose, because they are biologically suitable and they do not substantially influence the mechanical properties of the material.

Other biocompatible substances that may optionally be used as additives to the hydraulic cements are selected from the group consisting of calcium carbonate, calcium phosphate, apatite, fluorapatite, carbonates-apatites, and hydroxyapatite.

Dimension controlling phases, primarily calcium silicates and fumed silica (very finely grained silica), may be added. The function of such additives is to control the expansion occurring during curing, suitably such that the expansion is about 0.5-0.8% for orthopaedic applications or 0.3% for dental filling applications.

Other additives may be used to control the viscosity or workability (herein called water reducing agents) . Most preferred are organic polymers providing dispersion effects. These may e.g. be varieties of polycarboxylic acids or polyacrylic acids and superplasticisers.

The biocement slurry or paste may also contain an agent that accelerates or retards the hardening process of the calcium aluminate. Such accelerator or retarder components are well known in the field. Lithium chloride (LiCl) has been shown to be an especially suitable accelerator. Polysaccharide and other sugars have been recognised as usable retarders.

1. Biocompatible cement composition, comprising fixation grains (granules) in a matrix of hardened biocement.

2. Biocompatible cement composition according to claim 1, wherein the fixation grains are selected from a group comprising biological and biocompatible materials, metals and alloys thereof, ceramics and polymers, but are preferably selected from the group consisting of biocompatible materials, such as titanium, vitallium alloys of the Co—Cr—Mo—V system, stainless steels, Co—Cr alloys, alumina, zirconia, silicon nitride or SiAlONs, or biological materials, such as bone powder or chips.

3. Biocompatible cement composition according to claims 1, wherein the biocement comprises hydraulic ceramic powder binder phase.

4. Biocompatible cement composition according to claim 3, wherein the hydraulic binder phase is selected from the group consisting of calcium aluminate, calcium silicate, or a combination thereof.

5. Biocompatible cement composition according to claim 1, wherein the biocement comprises particles or powder of one or more non-hydraulic filler materials.

6. Biocompatible cement composition according to claim 5, wherein the non-hydraulic filler material comprises calcium titanate or any other ternary oxide of perovskite structure according to the formula ABO3, where O is oxygen and A and B are metals, or any mixture of such ternary oxides.

7. Biocompatible cement composition according to claim 1, wherein the biocement comprises particles or powder of one or more biocompatible materials selected from the group consisting of calcium carbonate, calcium phosphate, apatite, fluoroapatite, carbonates-apatites, and hydroxyapatite.

8. Biocompatible cement composition according to claim 1, wherein the biocement comprises powder has a grain size where more than 50 vol.%, preferably more than 80 vol.%, and most preferably more than 90 vol.% of the grains fall within the range 0.5-20 microns, and where 1-5 microns is the preferred size range.

9. Biocompatible cement composition according to claim 1, wherein the biocement slurry or paste comprises a component which accelerates (lithium chloride, LiCl) or retards (polysaccharide, sugar) the hardening process.

10. Biocompatible cement composition according to claim 1, wherein the biocement slurry or paste comprises a component which is a water reducing agent based on a compound selected from the group consisting of polycarboxylic acids, polyacrylic acids, and superplasticisers.

11. Biocompatible cement composition according to claim 1, wherein the biocement slurry or paste comprises expansion controlling additives selected from the group consisting of fumed silica, calcium silicate, or combinations thereof.

12. Method of filling a cavity in the skeleton, comprising mixing fixation grains (granules) and a biocement slurry or paste, introducing said fixation grains and biocement slurry or paste into the cavity, and allowing the formed mixture to harden.

13. Method according to claim 12, wherein the step of adding the fixation grains to the cavity includes packing them in said cavity.

14. Method according to claim 13, wherein the step of packing the fixation grains is performed by subjecting them to vibrations.

15. Method according to claim 12, wherein the fixation grains are selected from a group comprising biological and biocompatible materials, metals and alloys thereof, ceramics and polymers, but are preferably selected from the group consisting of biocompatible materials, such as titanium, vitallium alloys of the Co—Cr—Mo—V system, stainless steels, Co—Cr alloys, alumina, zirconia, silicon nitride or SiAlONs, or biological materials, such as bone powder or chips.

16. Method according to claim 12, wherein the biocement slurry or paste comprises a hydraulic ceramic powder binder phase.

17. Method according to claim 16, wherein the hydraulic binder phase is selected from the group consisting of calcium aluminate, calcium silicate, or a combination thereof.

18. Method according to claim 12, wherein the biocement slurry or paste comprises particles or powder of one or more non-hydraulic filler materials.

19. Method according to claim 18, wherein the non-hydraulic filler material comprises calcium titanate or any other ternary oxide of perovskite structure according to the formula ABO3, where O is oxygen and A and B are metals, or any mixture of such ternary oxides.

20. Method according to claim 12, wherein the biocement slurry or paste comprises particles or powder of one or more biocompatible materials selected from the group consisting of calcium carbonate, calcium phosphate, apatite, fluoroapatite, carbonates-apatites, and hydroxyapatite.

21. Method according to claim 12, wherein the biocement comprises powder having a grain size where more than 50 vol.%, preferably more than 80 vol.%, and most preferably more than 90 vol.% of the grains are within the range 0.5-20 microns, and where 1-5 microns is the preferred size.

22. Method according to claim 12, wherein the fixation grains have an irregular shape.

23. Method according to claim 12, wherein the biocement slurry or paste comprises a component which accelerates (lithium chloride, LiCl) or retards (polysaccharide, sugar) the hardening process.

24. Method according to claim 12, wherein the biocement slurry or paste comprises a component which is a water reducing agent based on a compound selected from the group consisting of polycarboxylic acids, polyacrylic acids, and superplasticisers.

25. Method according to claim 12, wherein the biocement slurry or paste comprises expansion controlling additives selected from the group consisting of fumed silica, calcium silicate, or combinations thereof.

26. Method according to claim 12, wherein the method is performed in one step, i.e. adding the fixation grains together with the biocement slurry or paste at the same time to said cavity.

27. Method according to claim 12, wherein the method is performed in two steps, i.e. first adding the fixation grains to the cavity and then adding the biocement slurry or paste to said fixation grains.
